(19) United States
(12) Patent Application Publication
(10) Pub. No.: US 2015/0289773 A1
(43) Pub. Date: Oct. 15, 2015

(54) HYPOTHENAR SENSOR

(71) Applicant: VitalSines Inc., Mississauga (CA)

(72) Inventor: Jesse Bruce Goodman, Mississauga (CA)

(21) Appl. No.: 14/746,287

(22) Filed: Jun. 22, 2015

Related U.S. Application Data
(62) Division of application No. 12/465,372, filed on May 13, 2009, now abandoned.
Application No. 12/465,372 is a division of application No. 13/761,725, filed on Feb. 7, 2013.

(51) Int. Cl. A61B 5/024; A61B 5/00

(52) U.S. Cl. CPC A61B 5/02438; A61B 5/742; A61B 5/6825; A61B 5/72; A61B 5/6833; A61B 2560/0412

(57) ABSTRACT

A device having an arterial pulse sensor that is adhered to the hypothenar region of a palm using an adhesive patch. The patch has an adhesive surface that is covered by a removable film with an outer portion and a central portion. Also disclosed is a method of detecting an arterial pulse by providing an arterial pulse sensor, placing the sensor on the hypothenar region of the palm of a hand, and receiving an arterial pulse signal from the sensor.

### CROSS REFERENCE TO RELATED APPLICATIONS

This application is a divisional application of pending Divisional application Ser. No. 13/761,725 filed Feb. 7, 2013 which is a Divisional of U.S. patent application Ser. No. 12/465,372 filed May 13, 2009, now abandoned.

### FIELD OF THE INVENTION

This invention relates to the field of medical devices, and in particular, sensors for detecting arterial pulse signals.

### BACKGROUND OF THE INVENTION

Arterial pulse sensors integrated with a motion sensor preferably satisfies several criteria to be used effectively in a commercial sense for motion tolerant biological signal utilization. The first criterion is that presence of the sensor does not lead to trauma to underlying tissue. A second criterion is that the sensor be able to provide adequate arterial pulse signal quality. A third criterion is that motion can be attenuated sufficiently for the arterial pulse signal to provide useful information during periods of motion.

Regions of the wrist that have been used for arterial pulse signal acquisition in the past, such as the area distal to the volar thumb over the radial artery and the dorsal wrist and forearm do not satisfy the three criteria noted above. The radial artery distal to the thumb is located between the skin and a bony surface, with little soft tissue to protect it. This can lead to trauma to the radial artery from a compressing sensor. The lack of soft tissue in this location results in physical motion of the skin surface associated with the arterial pulse. The dorsal wrist and forearm area is not associated with a closely associated arterial source. This results in a decrease in pulse signal quality.

### SUMMARY OF THE INVENTION

The hypothenar region of the palm is defined for the purposes of this application as the lateral part of the palm, from the wrist crease to the beginning of the little finger. Location of an arterial pulse sensor over the hypothenar region satisfies the three criteria noted above.

Location of a sensor over the hypothenar area of the palm is advantageous for acquisition of arterial pulse signals from the underlying ulnar artery. A data processing unit for an arterial pulse sensor located on the wrist may be integrated with a wristwatch in a convenient fashion. Integration of a motion sensor with an arterial pulse sensor permits recovery of the pulse signal when motion noise corrupts the pulse signal.

In one aspect, this invention relates to a device for contacting a hypothenar region of a palm comprising an arterial pulse sensor for detecting arterial pulse signals from an artery and attaching means for holding the sensor on the hypothenar region of the palm of a hand. The device can also have a data processing unit.

In another aspect, this invention relates to an adhesive patch for adhering a sensor to a hypothenar region of a palm, the adhesive patch comprising a first adhesive region for adhering the sensor to the patch and a second adhesive region for adhering the patch to the palm.

In a further aspect, this invention relates to a method of detecting an arterial pulse comprising the steps of providing an arterial pulse sensor, placing the sensor on the hypothenar region of the palm of a hand, and receiving an arterial pulse signal from the sensor.

In a still further aspect, this invention relates to a sensor module for contacting a hypothenar region of a palm. The sensor module can include a PPG sensor alone or a PPG sensor in combination with an accelerometer.

In a still further aspect, this invention relates to an adaptive filter used with three accelerometer axes for attenuation of motion noise with respect to a pulse sensor.

In a further aspect, this invention relates to a device that houses the sensor module.

In another aspect, this invention relates to an adhesive patch for adhering the data processing unit and securing the data processing unit to the hypothenar region of the palm. The patch has an adhesive surface that is covered by a removable film with an outer portion and a central portion. In one aspect, the patch is donut-shaped and has an elastomeric element that covers the central defect.

In a still further aspect, this invention relates to a kit with an arterial pulse sensor, a cable and an adhesive patch. In another aspect, the kit also has a motion sensor.

In a still further aspect, this invention relates to a method of tracking changes in mean blood pressure on a beat-to-beat basis by integrating ECG and arterial pulse sensors in a single enclosure for acquisition of arterial pulse wave velocity.

Other aspects and features of the present invention will become apparent to those ordinarily skilled in the art upon review of the following description of specific embodiments of a data processing unit, adhesives, and housing devices in conjunction with the accompanying drawing figures.

### BRIEF DESCRIPTION OF THE DRAWINGS

FIG. 1 is a drawing of a patch for a sensor, according to one embodiment of this invention.

FIG. 2 is a drawing of a glove and sensor, according to another embodiment of this invention.

FIGS. 3a and 3b are drawings of a spring mechanism for use with a sensor, according to yet another embodiment of this invention.

FIG. 4a is a drawing of the skin application side of a patch with two adhesive films according to another embodiment of this invention.

FIG. 4b is a drawing of the patch of FIG. 4a with the central film removed.

FIG. 4c is a drawing of the patch of FIG. 4b with a sensor according to this invention.

FIG. 4d is a drawing of the patch of FIG. 4c with the outer film removed.

FIG. 4e is a drawing of the non-skin application side of a patch according to another embodiment of this invention.

FIG. 5a is a cross-section of a patch, pad and sensor in use, according to one embodiment of this invention.

FIG. 5b is a cross-section of a patch, pad and sensor in use, according to another embodiment of this invention.

FIG. 6 is a perspective view of a wrist mounted data processing unit with the housing partially exposing a sensor, according to another embodiment of this invention.

FIG. 7 is a perspective top view of a device according to an embodiment of this invention.

FIG. 8 is a perspective bottom view of the device of FIG. 7.

FIG. 9 is a perspective top view of a device according to another embodiment of this invention.

Like reference numerals are used in different figures to denote similar elements.

### DETAILED DESCRIPTION OF THE INVENTION

In one embodiment, the present invention relates to an arterial pulse sensor located over the hypothenar region of a person's hand. The soft tissue over the hypothenar region cushions the pulse sensor and protects the underlying ulnar artery. The pulse sensor is a photoplethysmographic (“PPG”) sensor and is able to acquire good arterial pulse signal from this region. A PPG sensor uses light able to penetrate tissue to depths of several millimeters, sensing a pulse from the ulnar artery under the cushioning soft tissues of the hypothenar area.

Effectively positioning the pulse sensor over the hypothenar region requires that the sensor be applied to that area in a manner that maintains consistent contact between the sensor and the skin surface. A hold down force perpendicular to the skin surface is helpful in optimizing signal quality.

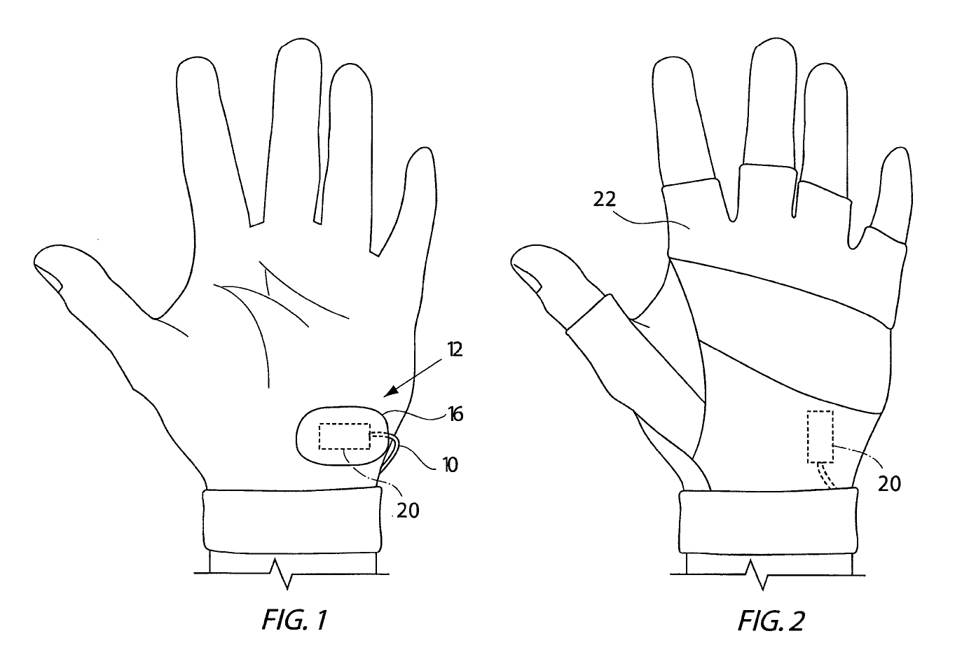

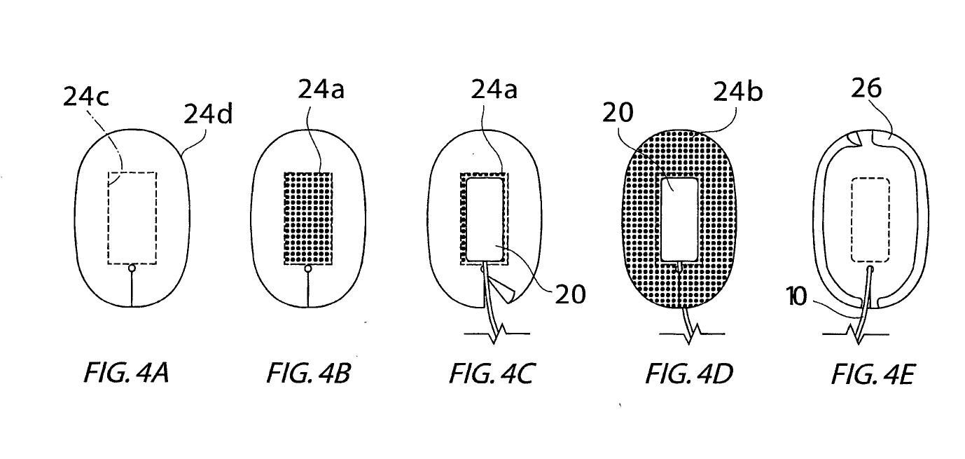

Referring initially to FIGS. 1 and 4a to 4e, the hold down force in one embodiment is provided by an adhesive patch indicated generally at 12 which is applied to the skin surface to hold the arterial pulse sensor 20 in place. The patch 12 has an adhesive surface on the skin application side which is covered by two separate removable films: a central film 14 and an outer film 16. The adhesive patch 12 can be made from materials that are non-toxic to the skin and maintain good adhesion over a period of several hours. The adhesive material used to hold breasts in an elevated position for women's fashion needs is one such material.

The central film 14 covers the central adhesive region 24a of the patch 12. Preferably, the central region 24a has the approximate shape and size of the “footprint” of the sensor 20. When the central film 14 is removed, the central adhesive region 24a is exposed and the sensor 20 can be adhered to the central adhesive region 24a to secure the sensor 20 more or less centrally on the patch 12.

The outer film 16 covers the outer adhesive region 24b which rings the central adhesive region 24a. The outer film 16 includes a slit 18 which runs across the film from the inner edge 24c to the outer edge 24d of the film 16. Preferably, a hole 28 is provided in the patch 12 passing through the central film 14 and the outer film 16 at the slit 18 for receiving a cable 10 for connecting the sensor 20 to a data processing unit (described below).

In an alternate embodiment, the patch 12 has a third separate and removable film 26 that encircles the perimeter of the patch 12. The film 26 has a degree of rigidity to it and helps frame the patch and hold its shape while it is being applied to a skin surface. Once the patch 12 has been applied to the skin surface, the film 26 can be removed to allow the patch 12 to more easily flex with the skin.

In operation, to apply the patch 12, the first step is to remove the central film 14 and adhere the sensor 20 onto the adhesive surface 24 or adhesive pad 32 at the location previously covered by the central film 14.

The second step is to feed a cable 10, connecting the sensor 20 to an associated processing device, through the hole 28 adjacent to the central portion of the adhesive patch. As shown in FIGS. 4a to 4e, the action of feeding the cable 10 through this hole 28 is facilitated by a slit 18 in the adhesive patch 12 extending from the hole 28 to the edge perimeter of the patch 12. This slit 18 can be lifted on one side by a user, to allow easy placement of the cable 10 through the hole 28.

The third step is to remove the outer film 16 covering the outer area of the adhesive patch 12 and place the patch over the hypothenar area, as shown in FIG. 1. The slit 18 discussed above has each of its edges firmly adherent to the skin.

Once the patch is secure, the fourth step is to remove the third removable film 26 if present. The cable 10 can be located above the patch 12 and can be connected to a processing and display apparatus located on the hand, wrist or forearm.

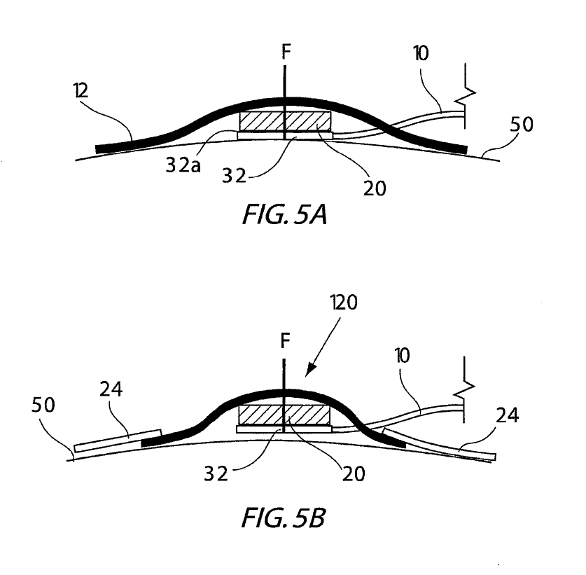

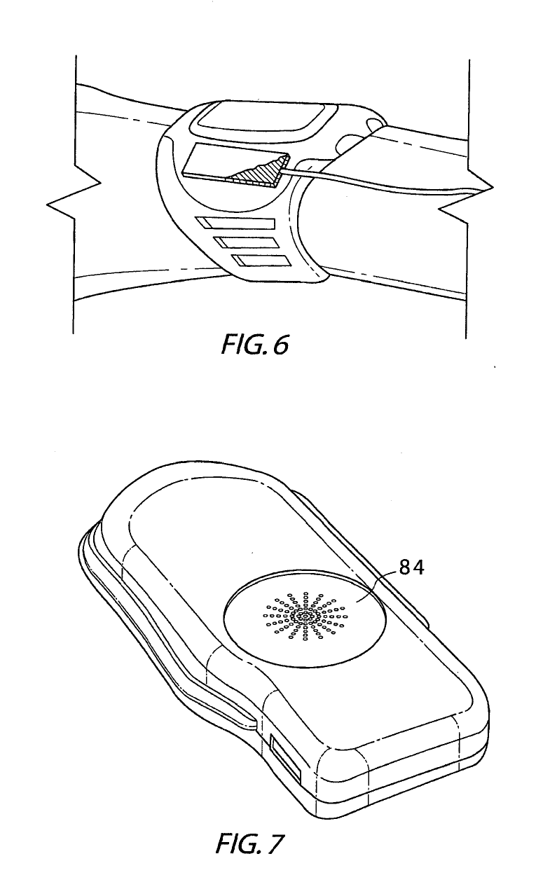

Referring to FIG. 5a, in a further embodiment, the patch 12 can be used with an adhesive foam pad 32 having approximately the same shape and size of the “footprint’ of the sensor 20. The pad 32 can be adhered to the adhesive surface 24a. The pad 32 has an adhesive surface 32a for adhering to the sensor 20 such that when the patch 12, pad 32 and sensor 20 are applied to the skin, the pad 32 is positioned between the patch 12 and the sensor 20. The sensor module 20 functions best when a force (F) perpendicular to the skin surface acts on the sensor to hold it firmly against the skin surface. As shown in FIG. 5a, the patch 12 adherent to the skin 50 surrounding sensor module 20 will exert force (F) on the sensor, perpendicular to the skin surface creating better skin-sensor contact.

Referring to FIG. 5b in another embodiment, the patch according to this invention consists of a donut-shaped adhesive 120 with the central defect defined by the adhesive 120 covered by an elastomeric element 40 attached to the adhesive 120. The elastomeric element 40 may be elastic fabric. Use of the donut shaped adhesive 120 permits the elastomeric element 40 to be bonded to the adhesive ‘donut ring’ in a firm manner. In one embodiment, an adhesive not suitable for direct application to the skin can be used. The elastomeric element 40 is adapted to exert adequate force on the sensor 20 perpendicular to the skin surface, and the adhesive substance bonding the ‘donut ring’ to the skin 50 not being disrupted by a distracting force perpendicular to the skin surface. In other terms, the force (F) perpendicular to the skin surface is maximized over the sensor 20 and minimized around the ‘donut ring’ through use of an elastomeric element 40 bonded to the ‘donut ring’ as shown in FIG. 5b.

Increased tension in a paper tape type patch material may lead to tearing; excessive tension in a plastic type patch will exert traction on the adhesive and pull the patch off the skin surface.

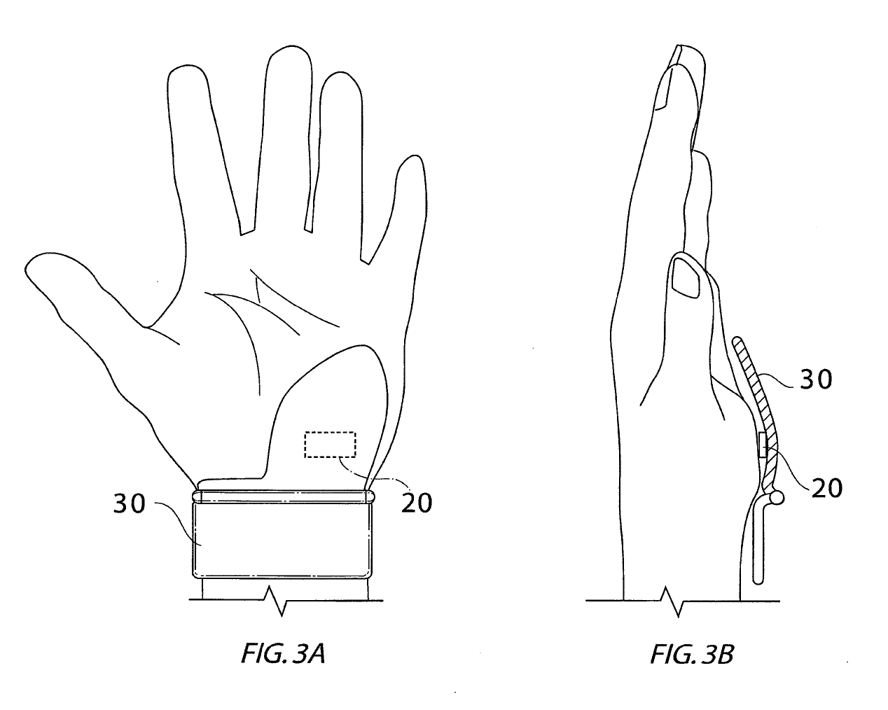

In another embodiment, the sensor 20 is integrated with a glove 22 (shown in FIG. 2). In a further embodiment, a spring loaded mechanism 30 (shown in FIGS. 3a and 3b) holds the sensor 20 in place on the skin.

In one embodiment, the sensor 20 can be used without an associated motion sensor to produce a pulse signal during periods when motion is absent. During motion, it is possible to use techniques that recover useful pulse signal information, without the need for a motion sensor. These techniques include time and frequency domain techniques which would be known to a person of ordinary skill in the art and in other embodiments, the glove 20 and mechanism 30 can be integrated with a garment such as an extension to a sleeve cuff. In a further embodiment, a band such as an elasticised fabric band can be used which extends across the palm and encircles the hand and/or the thumb.

In another embodiment, the sensor 20 can be used with a motion sensor to detect motion noise. The motion sensor can be an accelerometer. In one embodiment, the accelerometer and infrared sensors are two separate components placed on the same printed circuit board. The accelerometer is placed over the hypothenar region and can be used to attenuate motion noise corrupting the infrared sensor signal sufficiently to allow use of the arterial pulse signal for arterial pulse analysis purposes during times when the sensor is subject to motion. Effective motion noise attenuation can be related to both the cushioning effect of the soft tissues of the hypothenar area, damping motion of the skin surface associated with the arterial pulse, and optional motion noise attenuation through use of an adaptive filter using motion sensing to provide motion signals used as a noise reference signal.

Motion of the skin surface induced by the arterial pulse will produce pulse-like variations in the motion sensor, impairing its ability to act as a pure motion noise reference source. Motion sensing can be performed with various conventional technologies, including but not limited to, accelerometers and piezo sensors. Motion and arterial pulse sensors can be combined into a single pulse sensor on a single printed circuit board, so that motion of pulse and motion sensors is similar, i.e. they effectively experience the same motion. The motion of the accelerometer can then be used as an estimate of the motion noise included with the pulse signal.

Adaptive filters, using pulse and motion noise reference signals, can be of the form of Least Mean Squares (“LMS”), Recurrsive Least Mean Squares (“RMS”), RLS, Kalman or other similar adaptive filters. According to a method of this invention, signals from a three-axis accelerometer can be used as a single noise reference signal as follows. At each instant of time, the adaptive filter requires an input vector and target signal. An input vector is created by taking a window (or segment) of each of the accelerometer channels, that consists of a set of samples preceding and following the target signal. Specifically, let x1(n), x2(n), and x3(n) represent the three channels of the accelerometer and y(n) is the optical signal and is a separate input to the Kalman filter.

For example, suppose that one wishes to use segments of each of the three input channels that are 5 samples in duration. The input vector for the adaptive filter at time n is then defined as:

x(n)=[x1(n−2)x1(n−1)x1(n)x1(n+1)x1(n+2)x2(n−2)x2(n−1)x2(n)x2(n+1)x2(n+2)x3(n−2)x3(n−1)x3(n)x3(n+1)x3(n+2)]

The data processing unit can include a wireless device to send data to a wireless receiver. In one embodiment, data from the sensor is sent using Bluetooth technology to a cell phone, personal digital assistant, personal computer or other computer device. The data can then in turn be sent to an Internet server for further processing, storage and display. The processed data could follow a reverse path and be displayed on the screen of the data processing unit associated with the hypothenar sensor. Other prior art wireless strategies could be used to enable the above scenario.

A sensor attached to the skin through use of an adhesive patch according to this invention could incorporate a drug delivery system in the patch. The patch adhesive could be formulated to contain a pharmaceutically active substance. This substance could be a vitamin or any other transdermally acting substance. The substance could also be designed to improve skin and circulatory properties affecting sensor performance, such as a vasodilator substance to increase blood flow to the area underlying the patch.

As shown in FIG. 6, a sensor 20 used to detect the arterial pulse in the hypothenar area of the palm can be connected to a data processing unit housed in a wristwatch type device 60 using connectors (not shown) of various kinds. These connectors in various embodiments can be cables, ribbon connectors and other connector designs. The connector can provide removable attachment means to the wristwatch type device 60 or can be permanently attached to the wristwatch type device. If attached permanently, it may be possible to provide a housing 70, integrated with the wristwatch type device 60, to house the sensor 20 when not in use. The sensor 20, in one embodiment, would slide into the housing 70, located on the side of the wristwatch type device 60. The sensor 20 could be held in place in the housing 70 by various means, including pressure fit and locking clasp.

In a further embodiment, ECG signal acquisition can be used with hypothenar arterial pulse signal acquisition to calculate arterial pulse wave velocity. The time delay between a designated component of the ECG signal, such as the ECG signal ‘R’ wave, and a designated component of the arterial pulse signal, such as a point 50% up the ascending limb of the pulse signal, is termed pulse transit time (PTT). PTT can be used with the height of the individual being tested, to approximate the distance between the heart and the hypothenar area and calculate arterial pulse wave velocity (PWV). PWV is well correlated with mean blood pressure. PWV can be used to track changes in mean blood pressure on a beat-to-beat basis.

Use of PWV to track beat-to-beat changes in mean blood pressure has value for detection of conditions where blood pressure changes rapidly in response to certain provocative maneuvers, such as standing up from a sitting position. A drop in blood pressure related to a change in body orientation is known as an orthostatic drop. Conditions such as dehydration, medication side effects, neurological conditions and diabetes can produce an abnormal orthostatic response. An ability to follow PWV using easy to use acquisition hardware would be of medical benefit.

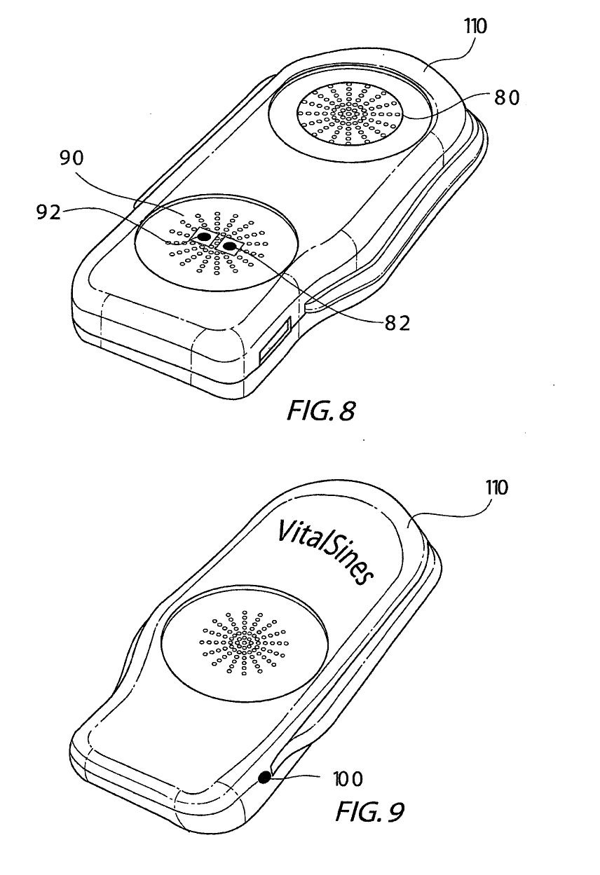

In another embodiment, a device is provided combining hypothenar arterial pulse capture as well as ECG signal acquisition in a single housing is adapted to sit over the hypothenar area. FIGS. 7 and 8 show a device with two ECG electrodes 80, 82 on one surface and one ECG electrode 84 on the other surface. One of the ECG electrodes 82 is integrated with an infrared arterial pulse sensor. The infrared pulse sensor in one embodiment may include an infrared LED 90 and a photodiode 92. The enclosure 110 may be held between an individual's two hands in order to obtain an ECG signal.

In another embodiment, a phono jack 100, a round opening in the enclosure 110 seen in FIG. 9, can be used to connect an external ECG lead for attachment to a point on the opposite side of the body, possibly the opposite wrist.

Display of the associated information in one embodiment could be through an LCD type display built into the enclosure. In another embodiment, display could be through use of a wireless connection to a cellular phone, personal computer, network or other processing and display means.

What is claimed is:

1. A method of detecting an arterial pulse comprising the steps of: providing an arterial pulse sensor; placing the sensor on the hypothenar region of the palm of a hand; and receiving an arterial pulse signal from the sensor.

2. The method of claim 1, further comprising the step of providing a data processing unit for receiving and processing the arterial pulse signal.

3. The method of claim 1, further comprising the step of providing a display device and displaying the processed signal on the device.
